(19) United States
(12) Patent Application Publication
(10) Pub. No.: US 2021/0007799 A1
(43) Pub. Date: Jan. 14, 2021

(54) Treatment Method and Medical Device

(71) Applicant: Alivas Inc., Tokyo (JP)

(72) Inventors: Tomoyuki Tajima, Tokyo (JP); Kazuyuki Saito, Chiba-shi (JP); Shohei Matsuhara, Chiba-shi (JP); Yoshiki Watanabe, Tokyo (JP)

(21) Appl. No.: 17/032,897

(22) Filed: Sep. 25, 2020

Related U.S. Application Data
(63) Continuation of application No. PCT/JP2019/013892, filed on Mar. 28, 2019.
Application No. PCT/JP2019/013892 is a continuation of application No. PCT/JP2018/012839, filed on Mar. 28, 2018.

(30) Foreign Application Priority Data
Mar. 1, 2019 (JP) 2019-037598

(51) Int. Cl. A61B 18/14; A61B 18/12; A61B 18/18

(52) U.S. Cl. CPC A61B 18/1492; A61B 2018/00494; A61B 18/1815; A61B 18/1206

(57) ABSTRACT

A treatment method and medical device for enhancing peristalsis are provided. The treatment method comprises enhancing the peristaltic movement of the gastrointestinal tract by performing a treatment to reduce the activity of the autonomic nerve in a blood vessel V having a surrounding nerve Sa innervating the gastrointestinal tract of the patient.

### CROSS-REFERENCE TO RELATED APPLICATION(S)

This application is a continuation of International Patent Application PCT/JP2019/013892, filed Mar. 28, 2019, which claims priority to PCT/JP2018/012839, filed Mar. 28, 2018 and claims priority to Japanese Patent Application No. 2019-037598, filed Mar. 1, 2019, the entire disclosures of which are hereby incorporated by reference.

### TECHNICAL FIELD

The present invention relates to a treatment method for enhancing the peristaltic movement of the gastrointestinal tract, and a medical device.

### BACKGROUND

Constipation is a common disease that is highly prevalent throughout the world, affecting some 25-30% of the population. Chronic constipation is defined as having less than three bowel movements per week with difficulty in defecating. Decreased frequency of defecation is known to cause symptoms of abdominal fullness, abdominal pain, dyspepsia and discomfort on defecation, frequent small bowel movements, and significantly impairs the patient's social activity and mental health.

Many patients with chronic constipation do not seek medical attention, but when they do, they are treated with medications aimed at normalizing the frequency of bowel movements and relieving the various abdominal symptoms of constipation. Drugs with a mechanism of promoting peristalsis and drugs with a mechanism of promoting secretion or inhibiting reabsorption from the gastrointestinal tract and softening and swelling the stool have been used in the past as therapeutic agents, and some have been newly developed (for example, see WO 2013-168671).

### SUMMARY

However, it is widely known that the majority of patients receiving such medication at medical institutions are not satisfied with their current treatment, and drug treatment is not a sufficient solution for the treatment of constipation.

There are two types of chronic constipation: primary (idiopathic) and secondary. Secondary constipation is caused by organic diseases such as colorectal cancer, drugs such as opioids used for pain relief, and systemic diseases such as neurological diseases and collagen diseases. Primary (idiopathic) constipation, on the other hand, can be divided into organic and functional constipation, depending on the presence or absence of organic abnormalities (such as dilatation) in the colon. Functional constipation without any organic abnormality in the colon is called “slow transit time constipation” when the peristalsis of the gastrointestinal tract is decreased, resulting in a delay in the passage of stool and causing constipation. For patients with severe constipation who do not respond to medical treatment, a total or partial resection of the colon may be recommended, but it is not widely accepted due to its invasive nature.

After diligent study, the inventors of the present invention have found an effective and minimally invasive method of treatment for various symptoms and diseases caused by a decrease or lack of peristalsis of the gastrointestinal tract.

The method of treatment according to one embodiment includes increasing peristaltic movement of the gastrointestinal tract by performing a procedure that reduces the activity of the autonomic nervous system in a blood vessel having a perineural nerve that innervates the patient's gastrointestinal tract.

According to the disclosure herein, the peristaltic motion of the gastrointestinal tract of a patient can be enhanced.

### BRIEF DESCRIPTION OF THE DRAWINGS

FIG. 1 shows a flowchart of the procedure of the treatment method according to the first embodiment;

FIG. 2 shows the blood vessel to which the treatment method is applied;

FIG. 3 shows a vertical cross-sectional view schematically showing a state in a blood vessel when the treatment method according to the first embodiment is performed;

FIG. 4 shows a cross-sectional view schematically showing a state in a blood vessel when the treatment method according to the first embodiment is performed;

FIG. 5 shows a configuration of a medical device according to a second embodiment;

FIG. 6 shows a flowchart showing a procedure of the treatment method according to the second embody;

FIG. 7 shows a part of cross-sectional view of the blood vessel to be treated;

FIG. 8 shows a cross-sectional view of an example for using a medical device according to the second embodiment;

FIG. 9 shows a perspective view showing an enlarged part of the treatment device according to first modification;

FIG. 10 shows a cross-sectional view showing an example for using the treatment device according to first modification;

FIG. 11 shows a cross-sectional view showing an example for using the treatment device according to second modification;

FIG. 12 shows a cross-sectional view showing an example for using the treatment device according to third modification;

FIG. 13 shows a cross-sectional view showing an example for using the treatment device according to fourth modification;

FIG. 14 shows a cross-sectional view showing an example for using the treatment device according to fifth modification; and

FIG. 15 shows a cross-sectional view showing an example for using the treatment device according to sixth modification.

### DETAILED DESCRIPTION

Embodiments of the present invention will be described with reference to the accompanying drawings. The following description is not intended to limit the significance of the technical scope or terms described in the claims. The dimensional ratios in the drawings are exaggerated for the sake of explanation and may differ from actual ratios. The range “X to Y” as shown herein means “X or greater, Y or less”.

### First Embodiment

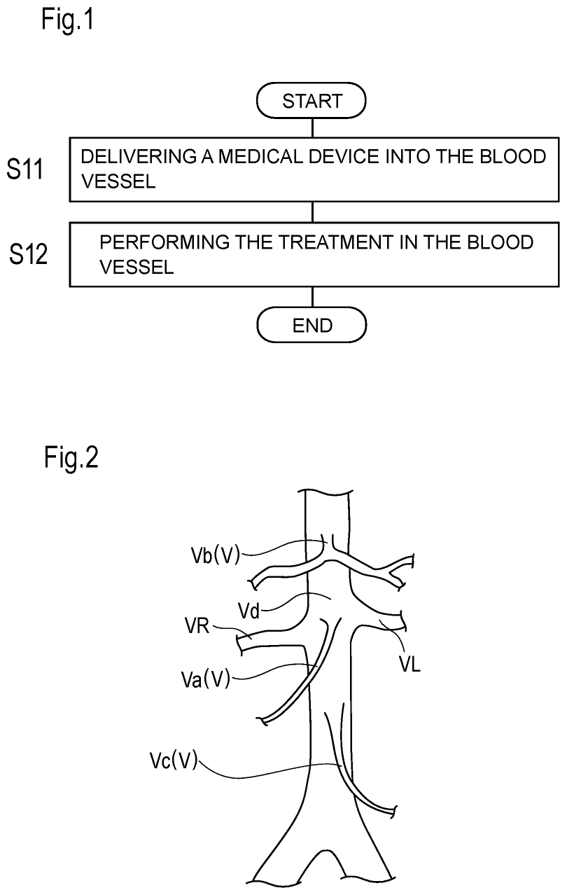

FIG. 1 illustrates in outline a procedure of the treatment method (hereinafter referred to as the “treatment method”) in the first embodiment by means of a flowchart. As shown in FIG. 1, the treatment method includes, in general, delivering a predetermined medical device into a blood vessel in which the treatment is performed (S11), and performing the treatment in the blood vessel (S12).

Referring to FIG. 2, the blood vessel V in which the treatment method is performed is described. In FIG. 2, the sign VR indicates the right renal artery and the sign VL indicates the left renal artery. The sign Va indicates the superior mesenteric artery, the sign Vb indicates the celiac artery, the sign Vc indicates the inferior mesenteric artery, and Vd indicates the aorta.

The blood vessel V to which the treatment method is applied is not particularly limited if it is possible to increase the peristaltic movement of the gastrointestinal tract of the patient (subject) by the application of the predetermined treatment (e.g., the impartation of energy, as described below) pertaining to the embodiment, but at least one of the superior mesenteric artery Va, the celiac artery Vb, and the inferior mesenteric artery Vc, for example, can be suitably selected.

The treatment site (range, location, etc.) to which the treatment is applied in the blood vessel V is not particularly limited as long as it is possible to enhance the peristaltic movement of the gastrointestinal tract. For example, within the blood vessel V, the treatment may be performed on any range (site) in the direction of travel of the blood vessel V (direction of extension) or on any range (site) in the circumferential direction (circumferential direction of the transverse section) of the blood vessel V. The treatment may be performed on the same blood vessel V. The treatment may be performed multiple times for multiple treatment sites of the same blood vessel V, or may be performed multiple times for any treatment site of a different blood vessel V. In other words, the treatment method according to this embodiment is not limited to the type of blood vessel to be treated, the treatment site of the blood vessel, the specific content of the treatment, the equipment and instruments used for the treatment, and the like, as long as it is possible to increase the peristaltic movement of the gastrointestinal tract of the patient by performing at least one treatment.

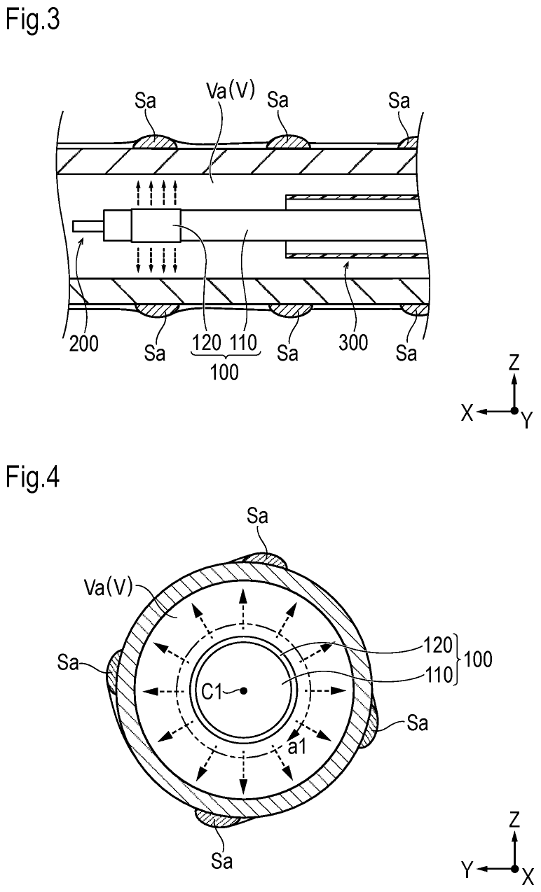

Next, referring to FIG. 3 and FIG. 4, the treatment method and the medical device 100 used for the treatment method in this embodiment will be described. In the following, an example of performing the treatment on the superior mesenteric artery Va as a blood vessel V to be treated will be described. The treatment procedure described below is only one example, and for example, for some procedures and procedures not specifically described, procedures known in the medical field can be employed as appropriate.

FIG. 3 is a cross-sectional view (longitudinal cross-sectional view) schematically illustrating the situation in the blood vessel V during implementation of the treatment method, and FIG. 4 is a cross-sectional view (transverse sectional view) schematically illustrating the situation in the blood vessel V during implementation of the treatment method. In FIG. 3 and FIG. 4, the running direction of the blood vessel V is indicated by arrow X, the depth direction orthogonal to the running direction of the blood vessel V is indicated by arrow Y, and the direction orthogonal to each of the running direction of the blood vessel V and the depth direction of the blood vessel V is indicated by arrow Z.

In the treatment according to the present embodiment, an operator such as a physician (hereinafter referred to as “operator”) performs a treatment for decreasing the activity of the autonomic nerve in a blood vessel V having a circumferential nerve (plexus) Sa that innervates the gastrointestinal tract of a patient, thereby enhancing the peristaltic motion of the gastrointestinal tract. By performing such a procedure, the operator facilitates alleviation of at least one symptom of abdominal distention, abdominal pain, perineal discomfort, frequent stools (at least one symptom group resulting from alleviation of constipation and/or abnormalities of peristaltic movement of the patient's gastrointestinal tract) caused by constipation and/or abnormalities of peristaltic movement of the gastrointestinal tract.

It is preferred to select at least one of the superior mesenteric artery Va, celiac artery Vb, and inferior mesenteric artery Vc as the blood vessel V to be treated, as described above. The surgeon then imparts energy to one or a plurality of surrounding nerves Sa as a procedure. Thereby, the surgeon impairs the perineural nerve Sa and increases peristaltic movement of the gastrointestinal tract by completely or partially blocking autonomic nerve transmission to the gastrointestinal tract by the perineural nerve Sa.

The following may be considered as the reason why the peristaltic motion of the gastrointestinal tract is activated by performing the treatment for decreasing the activity of the autonomic nerve in the blood vessel V having the surrounding nerve Sa. When the surrounding nerve Sa is innervated by the energy applied from the blood vessel V and the autonomic nerve transmission to the gastrointestinal tract by the surrounding nerve Sa is completely or partially blocked, the sympathetic is relatively weakened and the parasympathetic nervous system becomes dominant. In addition, the blocking of inhibitory nerve stimulation from the central nervous system causes the enteric nervous system, which autonomously controls gastrointestinal movement in the periphery, to become dominant, and the peristaltic movement of the gastrointestinal tract is activated. The activation of the peristaltic movement of the gastrointestinal tract promotes and normalizes colon transit time, thus promoting relief of at least one of the symptoms of abdominal bloating, abdominal pain, perineal discomfort, and frequent stools caused by constipation and/or abnormal intestinal peristaltic movement. In particular, the treatment method according to this embodiment can suitably promote relief of the symptoms of delayed transit time constipation in functional constipation in which there is no organic abnormality in the colon and in which the peristalsis of the colon is decreased and thus a delay in the transit time of the stool is observed, resulting in constipation.

The operator may perform a procedure using a given medical device 100, for example, as shown in FIG. 3 and FIG. 4. In particular, the operator may perform the procedure by actuating the medical device 100 with the medical device 100 including the energy-deliverable energy transfer member 120 disposed within the blood vessel V. In FIG. 3 and FIG. 4, the energy imparted by the medical device 100 is schematically illustrated by dashed arrows.

The medical device 100 may be a catheter device including an elongated catheter body 110 and an energy transfer member 120 disposed at a distal end portion of the catheter body 110 near an end portion in the insertion direction into the blood vessel V. The catheter body 110 includes a lumen (not shown) extending in the longitudinal direction (extending direction) of the catheter body 110, and a hub (not shown) disposed at the proximal end (near the end opposite to the insertion direction into the blood vessel V) of the catheter body 110. The medical device 100 is capable of passing a medical instrument, such as a guidewire 200, through a lumen and a hub, or flowing a liquid, such as a priming fluid. As a specific structure of the medical device 100, a known catheter device, for example, a therapeutic catheter device such as an ablation device, can be appropriately employed.

The energy transfer member 120 included in the medical device 100 may be configured to apply, for example, at least one of the group consisting of simple radio frequency, bipolar radio frequency, high-density focused ultrasound, ultrasound, microwave, light, heat, cold radiation, engineering therapy, magnetic, electrical, electromagnetic, cryotherapy, plasma, mechanical energy, chemical energy, kinetic energy, potential energy, nuclear energy, elastic energy, and hydrodynamic energy from the inside of the blood vessel V.

The operation of the energy transfer member 120 can be controlled, for example, via a controller (not shown). As the controller, for example, a known control device including a CPU and a storage unit can be used. The storage unit includes a ROM for storing various programs and data, a RAM for temporarily storing programs and data as a work area, a hard disk capable of storing various programs and data, and the like. A series of programs necessary for operation control of the medical device 100 can be stored in the storage unit. The energy transfer member 120 is electrically connected to a power supply unit (not shown), and can control ON/OFF of operation, the amount of energy applied to the blood vessel V, and the like via the power supply unit. Further, the transmission form of the operation command to the energy transmission member 120 can include, for example, a form by wire through a telecommunication line, a form by wireless without through a telecommunication line, a form which performs transmission based on an input from an operator or the like through an operation unit incorporated in a control device, a form which performs transmission based on an input from an external communication means or the like prepared as a device different from the control device, and the like, but the specific form is not particularly limited. In addition, the treatment according to the present embodiment may be performed by a medical device such as a treatment robot for replacing the work performed by the operator, for example. In this case, the treatment may be performed by controlling the treatment robot in a medical site such as an operating room or by controlling the treatment robot in a remote place.

The catheter body 110 of the medical device 100 is preferably configured to be movable to follow the curvature or flexion of the patient's blood vessel in order to deliver the energy transfer member 120 to the superior mesenteric artery Va, the celiac artery Vb, or the inferior mesenteric artery Vc. The catheter body 110 may employ various materials, structures, and the like used in known catheter devices having, for example, flexibility, torque transmission, kink resistance, and the like, in order to provide the above-described performance.

When inserting the medical device 100 into the blood vessel V, the operator can use the guide wire 200 and the guide catheter 300, for example, as shown in FIG. 3. As the guide wire 200 and the guide catheter 300, those known in the medical field can be used. The operator can also deliver the medical device 100 to a desired location (treatment site) of a blood vessel V (any of the superior mesenteric artery Va, the celiac artery Vb, and the inferior mesenteric artery Vc) via, for example, a femoral artery, an abdominal aorta, or the like.

After delivering the energy transfer member 120 to a desired location within the blood vessel V, as shown in FIG. 3, the operator actuates the energy transfer member 120 to apply energy from within the blood vessel V to the surrounding nerves Sa present outside the blood vessel V.

When performing a procedure, the operator can apply energy in at least one form, for example, in the entire circumferential direction of the blood vessel V (such a form that there is no intermittence along the inner wall of the blood vessel V), in a portion of the circumferential direction of the blood vessel V (such a form that only a portion of the inner wall of the blood vessel V is in a circumferential direction, for example, less than a half circumference), in a half circumference of the blood vessel V (such a form that the inner wall of the blood vessel V is in a shape that the inner wall of the blood vessel V is in a shape that it is continuous with a constant length in the circumferential direction, in a linear shape, and in a radial shape (such a direction that the inner wall of the blood vessel V is spaced radially outward. The operator can also perform a treatment by arbitrarily combining the above energy application forms.

When performing a treatment, the operator can adjust the output of the energy transfer member 120 so as to apply energy of, for example, 100° C. or less to the blood vessel V.

The operator can also control the range of application of energy in any range, e.g., from 0° to 360° of the cross-section of the blood vessel V, in the direction indicated by arrow al in FIG. 4, when performing the procedure. The operator can control the extent to which the surrounding nerve Sa is impaired within a range of 0% to 100% of the total surrounding nerve Sa and adjust the effect of increasing the peristaltic motion of the gastrointestinal tract.

The medical device 100 may also include one or more expandable structures near the distal end of the medical device 100. When the medical device 100 is configured in this manner, the operator can hold the medical device 100 in place in a direction parallel to the lumen of the blood vessel by expanding the expandable structure within the blood vessel V and temporarily fixing it to the blood vessel wall. The expandable structure may, for example, be configured integrally with the catheter body 110 or as a separate device from the catheter body 110. When the expandable structure is integrally formed with the catheter body 110, for example, a portion of the catheter body 110 is configured to be expandable. In this case, for example, the vicinity of the distal end portion of the catheter body 110 can be formed of a material having self-expandability or the like. Also, if the expandable structure is configured separately from the catheter body 110, for example, the expandable structure can be configured with a balloon catheter including a shaft having a lumen through which the medical device 100 can be inserted and projected distally, and a balloon that can be expanded and contracted.

When performing a procedure using the medical device 100, the operator may place the center position of the medical device 100, for example, at a position close to a predetermined position (a position deflected from the center position C1) in the circumferential direction of the blood vessel V from the center position C1 on the cross-section of the blood vessel V. By arranging the medical device 100 in this manner, the operator can effectively apply energy to the peripheral nerve Sa from any position in the circumferential direction of the blood vessel V. The medical device 100 can be appropriately provided with a positioning mechanism for arranging the medical device 100 as described above. The positioning mechanism may comprise, for example, a portion of the catheter body as well as the expandable structure, or may comprise a balloon.

The medical device 100 may also include one or more sensors that enable temperature measurement near the distal end of the medical device 100. When the medical device 100 is configured in this manner, the operator can control the output of energy based on the measurement result of the sensor. By appropriately controlling the output of the energy based on the measurement result of the sensor, the operator can prevent the effect of enhancing the peristaltic motion due to insufficient energy to be applied or the formation of a stenosis or the like in the blood vessel V due to excessive energy being applied to the blood vessel V. Note that there is no particular limitation on the type, arrangement position, number, and the like of the sensors.

Note that the medical device used in the treatment method according to the first embodiment is not particularly limited in specific structure, arrangement of members, and the like as long as energy can be applied in a blood vessel. For example, omission of the installation of the components (structures) of the medical device described by the illustration, use of additional components of other not specifically described, modifications to devices in the form other than catheter devices, and the like may be appropriately carried out.

Further, the specific procedure is not particularly limited as long as the treatment method at least includes enhancing the peristaltic movement by performing the treatment for reducing the activity of the autonomic nerve in a blood vessel having a peripheral nerve innervating the gastrointestinal tract of the patient.

### Second Embodiment

Next, a treatment method and a medical device according to a second embodiment of the present invention will be described. In the description of the second embodiment, the detailed description of the constituent members, the procedure, and the like already described in the first embodiment is omitted. Content, which is not specifically described in the description of the second embodiment, may be the same as the above-described embodiment.

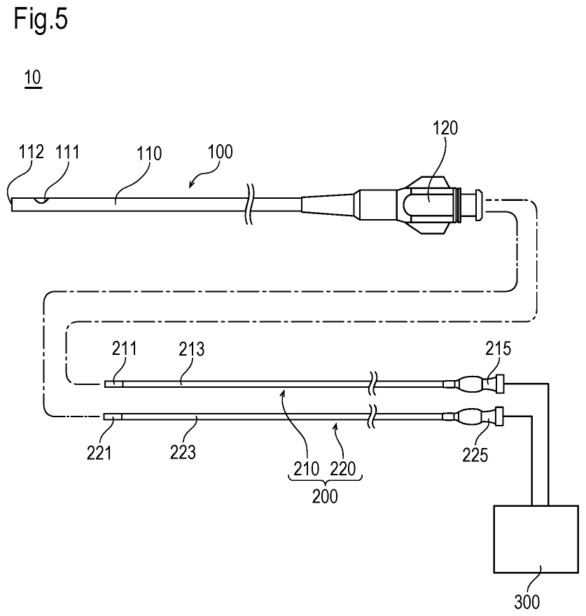

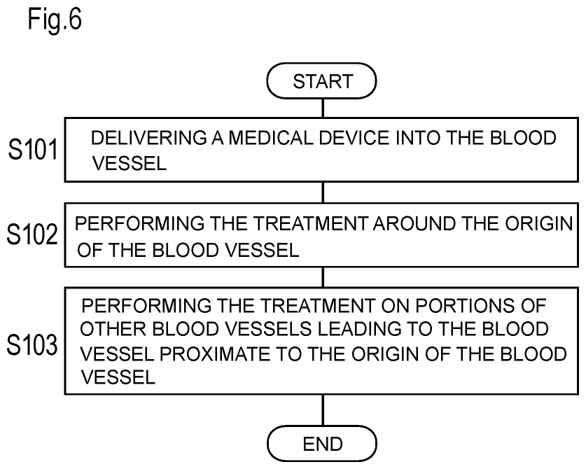

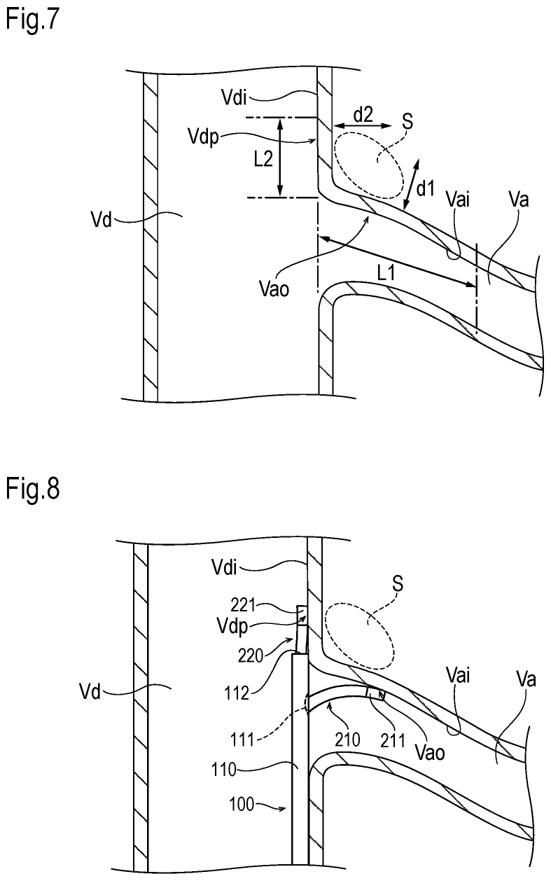

FIG. 5 is a schematic view of the medical device 10 according to the second embodiment, FIG. 6 is a flowchart showing a procedure of the treatment method according to the second embodiment, FIG. 7 is a cross-sectional view schematically showing a part of a blood vessel to be treated, and FIG. 8 is a cross-sectional view showing an example of use of the medical device 10 according to the second embodiment.

<Treatment method> Referring to FIGS. 6-8, a treatment method according to an embodiment will be described.

As shown in FIG. 6 and FIG. 8, the treatment methods generally include delivering predetermined treatment devices into a blood vessel (S101), performing treatment around the origin of the blood vessel (S102), and performing treatment on portions of other blood vessels leading to the blood vessel proximate to the origin of the blood vessel (S103).

In the procedure method in the second embodiment, as in the procedure method in the first embodiment, the operator increases the peristaltic movement of the gastrointestinal tract by performing a procedure to decrease the activity of the autonomic nervous system in the blood vessels having surrounding nerves (plexus) that control the gastrointestinal tract of the patient (the subject). By performing such a procedure, the operator can facilitate alleviation of at least one symptom of abdominal distention, abdominal pain, perineal discomfort, frequent stools (at least one symptom of a symptom group resulting from alleviation of constipation and/or abnormalities of peristaltic movement of the patient's gastrointestinal tract) caused by constipation and/or abnormalities of peristaltic movement of the gastrointestinal tract.

In the following description, an example is shown in which a blood vessel to be treated according to the embodiment is the superior mesenteric artery Va, and another blood vessel connected to the superior mesenteric artery Va is the aorta Vd.

As shown in FIG. 8, in the treatment method according to the embodiment, generally speaking, in the superior mesenteric artery Va having the surrounding nerve innervating the gastrointestinal tract of the patient, the peristaltic motion of the gastrointestinal tract is enhanced by performing the treatment of decreasing the activity of the autonomic nerve with respect to the periphery Vao of the origination portion of the superior mesenteric artery Va.

As shown in FIG. 8, the treatment method according to the embodiment includes performing treatment on a portion Vdp close to the origin of the superior mesenteric artery Va in the vicinity Vao of the origin of the superior mesenteric artery Va and the aorta Vd connected to the superior mesenteric artery Va.

Referring to FIG. 7, an example of a range of a treatment target region S (a region including one or a plurality of peripheral nerves) located outside each of the blood vessels Va and Vd will be described.

The treatment target region S preferably includes a range of 0 mm to 20 mm (a range indicated by the sign L1) along the extension direction of the superior mesenteric artery Va with reference to the opening of the superior mesenteric artery Va. By applying energy within the above-mentioned range of the superior mesenteric artery Va, it is possible to suitably suppress the transfer of energy to an organ located on the distal side of the superior mesenteric artery Va, for example, the pancreas or the duodenum. From the viewpoint of more reliably suppressing the transfer of energy to the organ located on the distal side of the superior mesenteric artery Va, it is particularly preferable that the application of energy from the superior mesenteric artery Va is performed only within the range of 0 mm to 20 mm along the extension direction of the superior mesenteric artery Va.

When energy is applied to the treatment target site S from the superior mesenteric artery Va side and the aorta Vd side as in the treatment method according to the present embodiment, the treatment target site S preferably includes a range of 0 mm to 100 mm (a range indicated by L2) along the extension direction of the aorta Vd with reference to the branch portion of the superior mesenteric artery Va. By applying energy from the aorta Vd side in such a range, it becomes possible to efficiently apply energy from both the aorta Vd side and the superior mesenteric artery Va side to the treatment target site S located outside each of the blood vessels Va and Vd.

The depth of energy from the superior mesenteric artery Va side (distance indicated by d1) is preferably at least 3 mm to 5 mm from the adventitia of the superior mesenteric artery Va. It is preferable that the depth of energy from the aorta Vd side (distance indicated by d2) is 3 to 5 mm from the adventitia of the aorta Vd. Surrounding nerves that lie outside the superior mesenteric artery Va lie 3-5 mm deeper from the adventitia in Vao around the origin of the superior mesenteric artery Va. More specifically, the surrounding nerves are present in bundles supported by connective tissue within the adipose tissue outside the superior mesenteric artery Va. Therefore, when energy is applied from the portion Vdp near the origin of the superior mesenteric artery Va in the vicinity Vao of the origin of the superior mesenteric artery Va and the aorta Vd, the peripheral nerve can be efficiently denervated by allowing the energy to reach the position of 3 to 5 mm from the adventitia of each blood vessel Va, Vd. The treatment target portion S can be set in an arbitrary range with respect to the outer circumferential direction of the superior mesenteric artery Va.

Depending on the blood vessel in which the denervation treatment is performed, organs may be present on the peripheral side of the blood vessel. For example, when the superior mesenteric artery Va is selected as a blood vessel to be treated, the pancreas and duodenum exist on the peripheral side of the superior mesenteric artery Va. It is preferable that the energy radiated from the superior mesenteric artery Va when the denervation is performed is not transmitted to the organ as much as possible in consideration of the influence on the health condition of the patient. According to the treatment method of the present embodiment, it is possible to suppress the transfer of energy to the pancreas and duodenum located on the distal side of the superior mesenteric artery Va.

Further, in the present embodiment, as shown in FIG. 8, the treatment for the treatment target site S radiates high frequencies from the interior-blood vessel wall Vai of Vao around the initiation portion of the superior mesial arterial Va and the bipolar electrodes 211 and 221 disposed at Vdi in the interior-blood vessel wall of the partial Vdp proximate to the initiation portion of the upper mesial arterial Va at the aorta Vd. Radio waves of 300 to 500 kHz, for example, can be selected as the high frequency.

However, the form of energy to be applied to the treatment target site S is not particularly limited as long as one or a plurality of peripheral nerves existing in the treatment target site S can be denervated, and is not limited to only high-frequency energy using the bipolar electrodes 211 and 221. For example, the form of energy of other can be selected from at least one of the group consisting of ultrasound, microwave, optical, thermal, cold radiation, engineering, magnetic, electric, electromagnetic, cryotherapy, plasmas, chemical energy, potential energy, nuclear energy, elastic energy, and hydrodynamic energy. Note that in the case where a microwave is used as the form of energy, the center frequency of the microwave can be set to any of 915 MHz, 2.45 GHz, 5.8 GHz, and 24.125 GHz, for example.

When applying energy to the treatment target region S, the operator supplies a current to each of the electrodes 211 and 221, and flows a current between each of the electrodes 211 and 221. While energy is applied from the electrodes 211 and 221 to the treatment target portion S, it is preferable to keep the electrodes 211 and 221 disposed in the vicinity of the treatment target portion S. Details of the medical device 10 (the treatment device 200 and the holding mechanism 100) used to apply energy to the treatment target site S will be described later.

In addition, it is preferable that the range (denervation range) in which energy is applied to the periphery Vao of the originating portion of the superior mesenteric artery Va is, for example, 50% or less in the outer circumferential direction of the superior mesenteric artery Va (180° or less in the circumferential direction on the cross section of the blood vessel). If the denervation range is 50% or more in the circumferential direction of the superior mesenteric artery Va, the increase of the peristaltic motion after denervation may be excessively promoted. Therefore, it is preferable to denervate the nerve in the above range.

<Medical Devices> As shown in FIG. 5 and FIG. 8, the medical device 10 generally includes a treatment device 200 including an energy transmission unit 211 capable of applying energy to the periphery Vao of the origination portion of the superior mesenteric artery Va having a peripheral nerve innervating the gastrointestinal tract of the patient, and a holding mechanism 100 capable of holding the energy transmission unit 211 to the inner wall Vai of the blood vessel of the periphery Vao of the origination portion of the superior mesenteric artery Va.

As shown in FIG. 5, the treatment device 200 may comprise one or more catheter devices 210, 220 comprising a first electrode 211 comprising a bipolar electrode and a second electrode 221 comprising a bipolar electrode.

In the description of the present embodiment, the side on which the electrodes 211 and 221 are arranged in each of the catheter devices 210 and 220 is the distal side, the side on which the hubs 215 and 225 are arranged in each of the catheter devices 210 and 220 is the proximal side, and the direction in which the catheter shafts 213 and 223 extend is the axial direction (longitudinal direction). In the description of the present embodiment, the distal end means a certain range including the distal end (most distal end) and its periphery, and the proximal end means a certain range including the proximal end (most proximal end) and its periphery.

As shown in FIG. 5, the first catheter device 210 includes a flexible catheter shaft 213, a first electrode 211 disposed at a distal end of the catheter shaft 213, and a hub 215 disposed at a proximal end of the catheter shaft 213. An electric wire for supplying an electric current to the first electrode 211 is inserted into the catheter shaft 213. The wires are drawn from the hub 215 and connected to the energy source 300. As the structure, material, and the like of each part of the first catheter device 210, for example, a structure of a catheter known in the medical field can be adopted.

The second catheter device 220 may be a device having substantially the same structure as the first catheter device 210. The second catheter device 220 includes a flexible catheter shaft 223, a second electrode 221 disposed at the distal end of the catheter shaft 223, and a hub 225 disposed at the proximal end of the catheter shaft 223. An electric wire for supplying an electric current to the second electrode 221 is inserted into the catheter shaft 223. The wires are drawn from the hub 225 and connected to the energy source 300. Like the first catheter device 210, the structure, material, and the like of each part of the second catheter device 220 can be appropriately adopted as a structure of a catheter known in the medical field.

The supply/stop of current to each of the electrodes 211 and 221, the adjustment of the current value, and the like can be controlled by the energy supply source 300. The energy supply source 300 includes a CPU and a storage unit. The storage unit may include a ROM for storing various programs and data, a RAM for temporarily storing programs and data as a work area, a hard disk capable of storing various programs and data, and the like. The storage unit can store a series of programs necessary for control such as supply/stop of current, adjustment of current value, and the like. The mode of transmitting and receiving the operation command of the energy supply source 300 may include, for example, a wired mode via a telecommunication line, a wireless mode without a telecommunication line, a mode of transmitting and receiving based on an input from an operator or the like via a controller, a mode of transmitting and receiving based on an input from an external communication means or the like prepared as a device different from the energy supply source 300, but the specific mode is not particularly limited. In addition, the treatment using each of the catheter devices 210 and 220 may be performed by a medical device such as a treatment robot that replaces the work performed by the operator. In this case, the treatment robot may be controlled in a medical site such as an operating room, or the treatment robot may be controlled in a remote place.

As shown in FIG. 5, the holding mechanism 100 comprises a guiding catheter comprising a flexible catheter shaft 110 and a hub 120 disposed at the proximal end of the catheter shaft 110.

The guiding catheter 100 has a distal end opening 112 opened at the distal end of the catheter shaft 110 and a side hole 111 formed at a predetermined position on the proximal end side of the distal end of the catheter shaft 110. Although not shown, a lumen communicating with the distal opening 112 and a lumen communicating with the side hole 111 may be provided inside the catheter shaft 110.

As shown in FIG. 8, the side hole 111 of the catheter shaft 110 can be used as a first guide portion for guiding the first catheter device 210 to the superior mesenteric artery Va. Further, as shown in FIG. 8, the distal end opening 112 of the catheter shaft 110 can be used as a second guide portion for guiding the second catheter device 220 to the aorta Vd.

When performing a procedure using each of the catheter devices 210 and 220, the operator projects the first electrode 211 located at the distal end of the first catheter device 210 from the side hole 111 of the catheter shaft 110 into the superior mesenteric artery Va with the catheter shaft 110 of the guiding catheter 100 in contact with the inner wall Vdi of the aorta Vd. The operator also causes the second electrode 221 located at the distal end of the second catheter device 220 to protrude from the aorta Vd from the distal end opening 112 of the catheter shaft 110. The operator can guide each of the electrodes 211 and 221 to a predetermined position at the time of performing the treatment on the treatment target portion S by performing the above-described operation. In addition, the operator can maintain the position of each electrode 211, 221 by maintaining the catheter shaft 110 in contact with the inner wall Vdi of the aorta Vd while performing the treatment by each electrode 211, 221.

In the present embodiment, since the form in which energy is applied to the treatment target portion S from each of the electrodes 211 and 221 constituting the bipolar electrode is adopted, there are the following advantages. (i) Since it is possible to apply energy from the blood vessels of both the superior mesenteric artery Va and the aorta Vd to the treatment target site S located between the blood vessels Va and Vd, it is possible to efficiently denervate the surrounding nerves existing in the treatment target site S. (ii) The range of influence of the energy radiated from each electrode 211, 221 is limited to a certain range between and around each electrode 211, 221. Therefore, it is possible to locally denervate a partial region in the patient's body. (iii) When a device capable of radiating an electromagnetic wave such as an antenna (e.g., a microwave) is mounted on a catheter device, it is possible to deepen the depth of energy penetration or to apply energy to the outside of a blood vessel in a state where it is not in contact with the inner wall of the blood vessel. However, since it is difficult in terms of technology to have directionality in the radial direction of the electromagnetic wave, the catheter device needs to be designed so that the catheter device can locally irradiate the electromagnetic wave to any position in the circumferential direction of the blood vessel. This complicates the apparatus configuration of the catheter device. On the other hand, according to the catheter devices 210 and 220 in which the electrodes 211 and 221 constituting the bipolar electrodes are mounted, there is no need to provide a mechanism for perfusing the fluid, a mechanism for adjusting the radiation direction of the energy, and the like, so that the configuration of the apparatus can be simplified. It should be noted that the treatment device (catheter device) may be configured by, for example, a single catheter device including an antenna element capable of radiating electromagnetic waves (e.g., microwaves). In this case, the catheter device may include a single catheter shaft in which an antenna element is disposed at the distal end, and may be configured to be able to apply energy to the periphery Vao of the origination portion of the superior mesenteric artery Va.

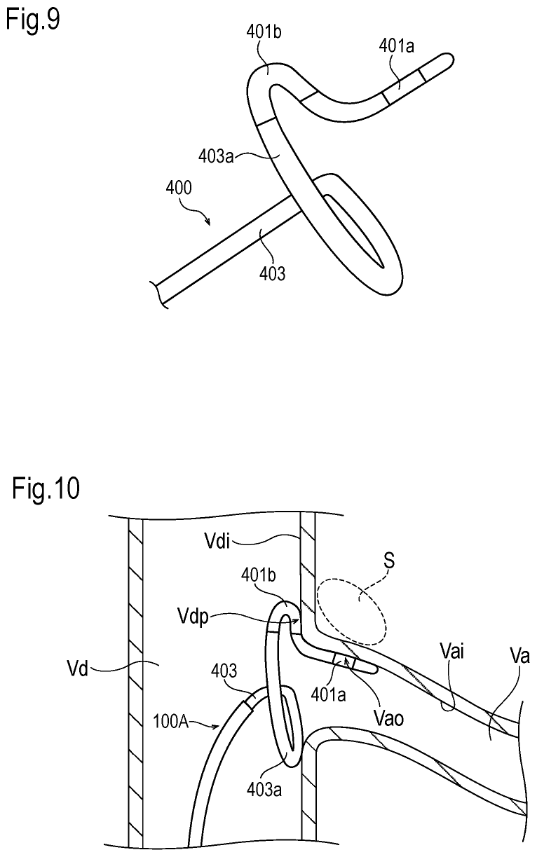

Next, each modification of the second embodiment will be described. As will be described below, the treatment methods and the medical devices according to the second embodiment are not limited to a specific content as long as at least the treatment for reducing the activity of the autonomic nerve with respect to the vicinity of the origin of the blood vessel can be performed in the blood vessel having the peripheral nerve innervating the gastrointestinal tract of the patient. In the description of the modification, the description of Content described above in the second embodiment is omitted. In addition, unless otherwise stated, the constituent members and the like of the respective modified examples can be the same as those of the above-described embodiment.

<First Modification> As shown in FIG. 9 and FIG. 10, the treatment device may be configured by, for example, a single catheter device 400.

The catheter device 400 according to the first modification includes a catheter shaft 403 and first and second electrodes 401a and 401b, which are disposed at different positions in the longitudinal direction of the catheter shaft 403 and constitute bipolar electrodes, respectively.

As shown in FIG. 10, the catheter shaft 403 is configured to be able to limit the insertion length by which the distal end portion of the catheter shaft 403 is inserted into the superior mesenteric artery Va by contacting the inner wall Vdi of the aorta Vd in a state in which the first electrode 401a is in contact with the inner wall Vai of the superior mesenteric artery Va and in a state in which the second electrode 401b is in contact with the inner wall Vdi of the aorta Vd.

The catheter shaft 403 has a distal end portion in which the first electrode 401a is disposed, and a curved portion 403a which is connected to the proximal end side of the distal end portion and in which the second electrode 401b is disposed. The curved portion 403a formed on the catheter shaft 403 constitutes a holding mechanism capable of holding the first electrode 401a against the inner wall Vai of the blood vessel in the vicinity Vao of the origin of the superior mesenteric artery Va.

As shown in FIG. 10, the curved portion 403a is shaped so as to form an outer shape larger than the entrance of the superior mesenteric artery Va. Therefore, when the energy is given to the treatment target site S, the distal end portion of the catheter shaft 403 is disposed in the vicinity of the origin periphery Vao of the superior mesenteric artery Va. Therefore, the catheter device 400 can locally apply energy to the treatment target site S while holding the first electrode 401a and the second electrode 401b on the inner walls Vai and Vdi of the blood vessels Va and Vd. As shown in FIG. 10, delivery of the catheter device 400 to the aorta Vd can be performed using a guiding catheter 100A known in the medical field.

The catheter shaft 403 is shaped so as to have the shape shown in FIG. 9 in a natural state in which no external force is applied. Although FIG. 9 shows an example in which a part of the catheter shaft 403 is formed in a circular shape, the shape of the catheter shaft 403 in the natural state is not particularly limited as long as the part of the catheter shaft 403 contacts the aorta Vd as shown in FIG. 10 and the insertion length at which the distal end of the catheter shaft 403 is inserted into the superior mesenteric artery Va can be limited. For example, a portion of the catheter shaft 403 may be shaped to have a polygonal shape or an elliptical shape. The position at which the electrodes 401a and 401b are arranged in the catheter shaft 403 is not particularly limited as long as energy can be applied to the treatment target site S arranged between the electrodes 401a and 401b.

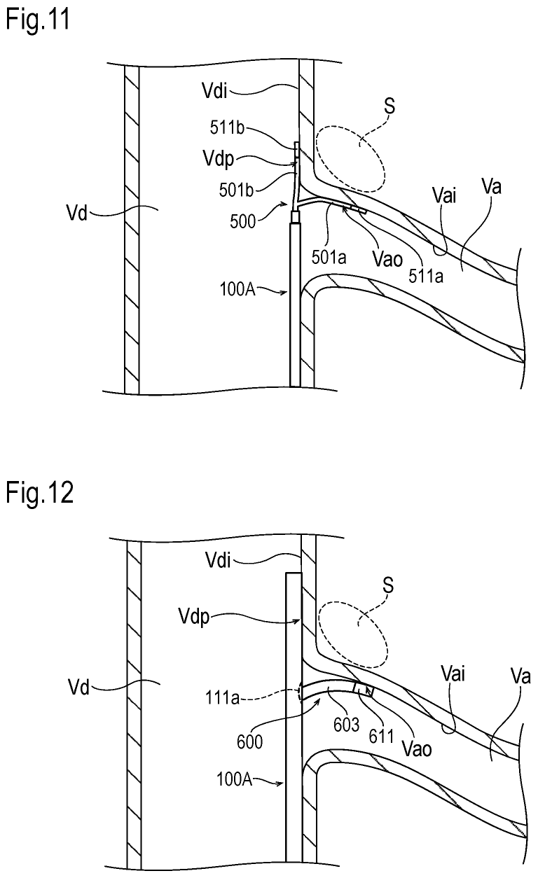

In the first modification shown in FIG. 9 and FIG. 10, a catheter device 400 including bipolar electrodes 401a and 401b disposed on a catheter shaft 403 is shown. However, the energy transfer member that applies energy to the treatment target portion S is not limited to only the bipolar electrode. For example, the energy transfer member may comprise an antenna element capable of radiating electromagnetic waves, e.g., microwaves. When an antenna element is used as the energy transfer member, for example, the antenna element can be disposed at a position where the first electrode 401a is disposed on the catheter shaft 403. Even in the event where the catheter device includes the antenna element, similarly to the catheter device 400 of the first modification, the insertion length by which the distal end portion of the catheter shaft 403 is inserted into the superior mesenteric artery Va can be limited by bringing a portion of the catheter shaft 403 into contact with the inner wall Vdi of the aorta Vd.

<Second Modification> As shown in FIG. 11, the catheter device 500 may have, for example, a structure in which the catheter shaft is bifurcated. A first electrode 511a may be disposed at the distal end of the bifurcated side 501a of the catheter shaft. A second electrode 511b may be disposed at the distal end of the other bifurcated side 501b of the catheter shaft.

<Third Modification> As shown in FIG. 12, the catheter device 600 may comprise, for example, a single catheter device including an electrode 611 disposed at the distal end of the catheter shaft 603. In this case, the electrode 611 can be formed of, for example, a unipolar electrode or an antenna element capable of radiating microwaves. The guiding catheter 100A used for delivery of the catheter device 600 can be used as a holding mechanism for holding the position of the electrode 611 during treatment with the electrode 611 by being brought into contact with the inner wall Vdi of the aorta Vd. In addition, the guiding catheter 100A may be provided with a side hole 111a for guiding the catheter device 600 into the superior mesenteric artery Va.

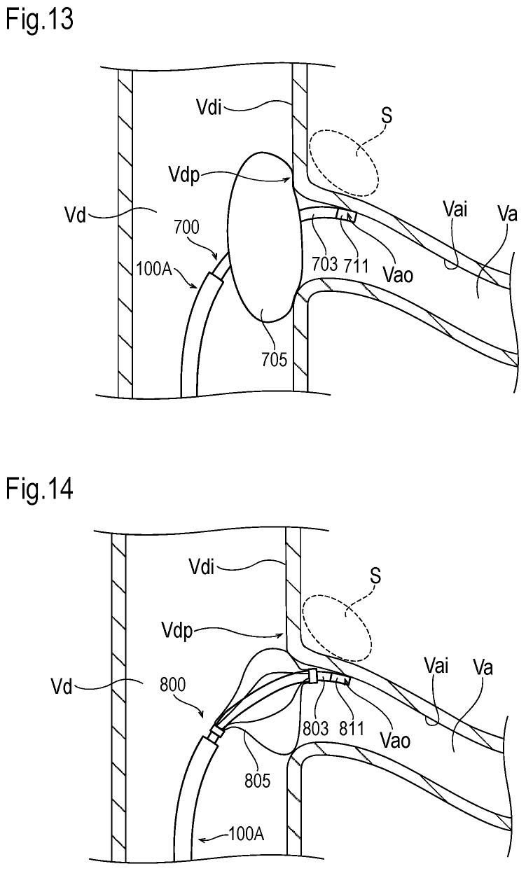

In the third modification shown in FIG. 12, the catheter device 600 and the guiding catheter 100A are formed of different members. Therefore, when guiding the catheter device 600 into the superior mesenteric artery Va, the operator delivers the catheter device 600 through a lumen (not shown) provided in the guiding catheter 100A. The catheter device 600 may be integrally formed with the guiding catheter 100A.

<Fourth Modification> As shown in FIG. 13, the catheter device 700 can be configured by a single catheter device including, for example, an electrode 711 disposed at the distal end of the catheter shaft 703 and a balloon 705 functioning as a holding mechanism. When energy is applied from the electrode 711, the balloon 705 can be held against the inner wall Vdi of the aorta Vd by expanding the balloon 705. This makes it possible to limit the insertion length by which the distal end portion of the catheter shaft 703 is inserted into the superior mesenteric artery Va.

The electrode 711 can be formed of, for example, a unipolar electrode or an antenna element capable of radiating microwaves. As the balloon 705, a known balloon that can be expanded and contracted in accordance with supply and discharge of a fluid can be used. Note that the specific shape, position, size, and the like of the balloon 705 are not particularly limited. The balloon 705 may be configured separately from the catheter shaft 703.

<Fifth Modification> As shown in FIG. 14, the catheter device 800 can be configured as a single catheter device including, for example, an electrode 811 disposed at the distal end of the catheter shaft 803, and a basket structure 805 functioning as a holding mechanism. Upon application of energy from the electrodes 811, the basket structure 805 can be held against the inner wall Vdi of the aorta Vd by expanding the basket structure 805. This makes it possible to limit the insertion length by which the distal end portion of the catheter shaft 803 is inserted into the superior mesenteric artery Va.

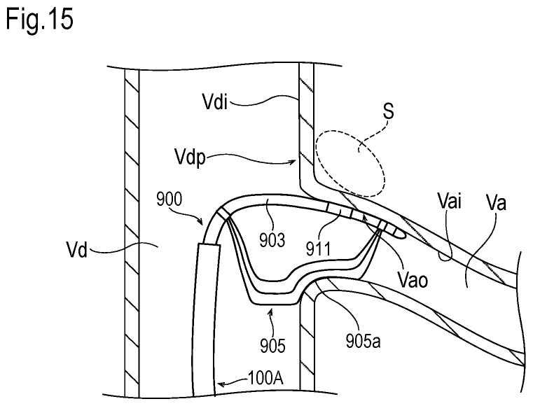

The electrode 811 can be formed of, for example, a unipolar electrode or an antenna element capable of radiating microwaves. As the basket structure 805, for example, a basket structure composed of a metal material having self-expandability (for example, a known titanium-based alloy) or a shaped resin material (for example, a known shape memory polymer) can be used. Note that the specific shape, position, size, and the like of the basket structure 805 are not particularly limited.

<Sixth Modification>As shown in FIG. 15, the catheter device 900 can be configured as a single catheter device including, for example, an electrode 911 disposed at the distal end of the catheter shaft 903, and a support structure 905 functioning as a holding mechanism. When energy is applied from the electrode 911, the support structure 905 is deformed into the shape shown in FIG. 12, whereby at least a part 905a of the support structure 905 can abut against the inner wall Vdi of the blood vessel of the aorta Vd and around the entrance of the superior mesenteric artery Va. This makes it possible to limit the insertion length by which the distal end portion of the catheter shaft 903 is inserted into the superior mesenteric artery Va.

The electrode 911 can be formed of, for example, a unipolar electrode or an antenna element capable of radiating microwaves. The support structure 905 can be composed of, for example, a plurality of wires shaped in the shape shown in FIG. 15. The support structure 905 may be formed of a metal material having self-expandability (for example, a known titanium-based alloy) or a shaped resin material (for example, a known shape memory polymer). The specific shape, position, size, and the like of the support structure 905 are not particularly limited as long as a part of the support structure 905 can be brought into contact with the peripheral portion around the entrance of the superior mesenteric artery Va.

Although the treatment method and the medical device according to the present invention have been described above through the embodiments, the present invention is not limited to content described in the specification, and can be modified as appropriate based on the description of the claims.

The structures described in each embodiment and each modification can be appropriately combined as long as the essential effects of the invention can be exhibited.

The medical device used in the treatment method according to the first embodiment is not particularly limited in specific structure, arrangement of members, and the like as long as energy can be applied in a blood vessel. For example, omission of the installation of the components (structures) of the medical device described by the illustration, use of additional components of other not specifically described, modifications to devices in the form other than catheter devices, and the like may be appropriately carried out. The specific procedure of the treatment method according to the first embodiment is not particularly limited as long as it includes at least enhancing the peristaltic movement of the gastrointestinal tract by performing a treatment for reducing the activity of the autonomic nerve in a blood vessel having surrounding nerves innervating the gastrointestinal tract of the patient.

The material, shape, size, arrangement, connection structure between members, and the like of each member constituting the treatment device (catheter device) and the holding mechanism according to the second embodiment are not particularly limited as long as the effect of the present invention is exhibited, and can be arbitrarily changed and substituted. In addition, any component or the like not specifically described in the specification can be appropriately added to the treatment device (catheter device) and the holding mechanism, and additional components described in the specification can be appropriately omitted. In addition, any procedure not specifically described in the specification can be appropriately added to the treatment method, and omission of the additional procedure described in the specification can also be appropriately performed. As for the treatment method, as long as the effect of the invention can be exhibited, the order of the procedures can be changed as appropriate. The blood vessels to be treated are not limited to those described in the embodiment.

### REFERENCE LIST

100: Medical devices (catheter devices)
110: Catheter body
120: Energy transfer member
200: Guidewire
300: Guide catheters
10: Medical devices
100,100A: guiding catheter (holding mechanism)
110: Catheter shaft
111: Side hole
112: Tip opening
120: Hub
200: Treatment device
210: First catheter device
211: First electrode (energy transfer unit)
213: Catheter shaft
215: Hub
220: Second catheter device
221: Second electrode (energy transfer unit)
223: Catheter shaft
225: Hub
300: Energy source
400,500,600,700,800,900: Catheter device
705 Balloon: (holding mechanism)
805 Basket structure: (holding mechanism)
905 Support structure: (holding mechanism)
V: Blood vessel
Va: superior mesenteric artery (blood vessel)
Vao: Around the origin of the superior mesenteric arteries
Vai: Inner wall of blood vessel
Vb: celiac artery (blood vessel)
Vc: inferior mesenteric artery (blood vessel)
Vd: aorta (other blood vessels)
Vdp: Proximate to the origin portion
Vdi: Inner wall of other blood vessels
S: Site to be treated

What is claimed is:

1. A treatment method comprising a treatment to increase peristaltic movement of a gastrointestinal tract of a patient by performing a procedure to decrease activity of autonomic nerves in a blood vessel having surrounding nerves that innervate the gastrointestinal tract, thereby increasing the peristaltic movement of the gastrointestinal tract.

2. The treatment method according to claim 1, wherein the treatment promotes relief of one or more of constipation abdominal bloating, abdominal pain, perineal discomfort, and frequent stools caused by abnormal peristalsis of the gastrointestinal tract in the patient.

3. The treatment method according to claim 1, wherein performing the treatment promotes relief of symptoms of slow transit time constipation.

4. The treatment method according to claim 1, wherein the blood vessel is at least one of a superior mesenteric artery, a celiac artery, and an inferior mesenteric artery,
wherein the treatment impairs the surrounding nerves by imparting energy to one or a plurality of the surrounding nerves and increases the peristaltic movement of the gastrointestinal tract by completely or partially blocking autonomic nerve transmission to the gastrointestinal tract by the surrounding nerves.

5. The treatment method according to claim 4, wherein the treatment is performed by activating a medical device disposed in the blood vessel, the medical device comprising a long catheter body and an energy transmitting member disposed at a tip of the long catheter body and capable of imparting energy.

6. The treatment method according to claim 4, wherein the treatment includes imparting the energy as one or more of simple radiofrequency, bipolar radiofrequency, high-density focused ultrasound, ultrasound, microwave, optical, thermal, cold radiation, engineering therapy, magnetic, electrical, electromagnetic, cryotherapy, plasma, mechanical energy, chemical energy, kinetic energy, potential energy, nuclear energy, elastic energy, and hydrodynamic energy.

7. A treatment method for increasing peristaltic movement of a gastrointestinal tract of a patient by performing a treatment to decrease activity of autonomic nerves around an origin of a blood vessel having surrounding nerves that innervate the gastrointestinal tract of the patient, thereby increasing peristaltic movement of the gastrointestinal tract.

8. The treatment method according to claim 7, wherein the treatment is performed on an area around the origin of the blood vessel and on a portion of another blood vessel connected to the blood vessel, the portion being adjacent to the origin of the blood vessel.

9. The treatment method according to claim 8, wherein the blood vessel is at least one of a superior mesenteric artery, a celiac artery, and an inferior mesenteric artery, wherein the other blood vessel is an aorta.

10. The treatment method according to claim 9, wherein a perimeter of the origin includes a range of 0 to 20 mm along a direction of extension of the blood vessel from the origin of the blood vessel, and the portion of the other blood vessel proximate to the origin includes a range of 0 mm to 100 mm along the direction of extension of the other blood vessel.

11. The treatment method according to claim 8, wherein the treatment includes radiating high-frequency waves from bipolar electrodes disposed in an inner wall of the blood vessel around the origin of the blood vessel and in the inner wall of the portion of the other blood vessel proximate to the origin of the blood vessel.

12. The treatment method according to claim 8, wherein the treatment includes radiating microwaves from an antenna element disposed around the origin of the blood vessel.

13. A medical device comprising:
a treatment device which includes an energy delivery unit configured to impart energy to an area around an origin of a blood vessel with surrounding nerves that innervate a gastrointestinal tract of a patient; and
a holding mechanism configured to hold the energy delivery unit to an inner wall around the origin of the blood vessel.

14. The medical device according to claim 13, wherein the energy delivery unit comprises bipolar electrodes disposed in the inner wall of the blood vessel around the origin of the blood vessel and in an inner wall of a portion of the blood vessel proximal to an origin of another blood vessel leading to the blood vessel,
wherein the holding mechanism is further configured to limit an extent to which the bipolar electrodes impart energy to the area around the origin of the blood vessel by holding the bipolar electrodes at least against the inner wall of the other blood vessel.

15. The medical device according to claim 13, wherein the treatment device further comprises one or more catheter devices with a first electrode and a second electrode comprising a bipolar electrode,
wherein the holding mechanism is a guiding catheter having a first guide portion guiding the first electrode to the blood vessel and a second guide portion guiding the second electrode to the other blood vessel.

16. The medical device according to claim 14, wherein the treatment device further comprises a catheter device having a catheter shaft, a first electrode and a second electrode disposed at different positions in a longitudinal direction of the catheter shaft and respectively configured to the bipolar electrode,
the holding mechanism comprises a portion of the catheter shaft,
the catheter shaft is inserted into the blood vessel with a tip of the catheter shaft in contact with the first electrode in contact with the inner wall of the blood vessel and the second electrode in contact with the inner wall of the other blood vessel, and
an insertion length is configurable to be limited.

17. The medical device according to claim 16, wherein the catheter shaft has the tip to which the first electrode is disposed, and a curved portion connected to a base side of the tip and to which the second electrode is disposed, and
the curved portion is shaped to form an outline larger than an entrance to the blood vessel.

18. The medical device according to claim 13, wherein the treatment device comprises a catheter device having a catheter shaft, and an antenna element capable of emitting microwaves disposed at a longitudinal end of the catheter shaft,
the holding mechanism comprises a portion of the catheter shaft, and
the catheter shaft is configured to limit a length of insertion of a tip of the catheter shaft into the blood vessel by bringing the antenna element in contact with the inner wall around the origin of the blood vessel and at least partially in contact with an inner wall of another blood vessel connected to the blood vessel.

19. The medical device according to claim 18, wherein the catheter shaft has a curved portion, which is connected to a base side of the tip where the antenna element is disposed, and
wherein the curved portion is shaped to form an outline larger than an entrance to the blood vessel.

20. The medical device according to claim 13, the treatment device comprises a catheter device comprising a catheter shaft with an antenna element for emitting microwaves,
wherein the holding mechanism is separate or integral to the catheter device.
